SEQUENCE LISTING (27 sequences)

SEQ ID NO: 1 - protein, 4 aa, Artificial Sequence
  Xaa is a variable which may be A,R,N,D,C,Q,E,G,H,I,L,K,M,F,P,S,T,W,Y,V This is a synthetically prepared peptide
ETXV

SEQ ID NO: 2 - protein, 4 aa, Artificial Sequence
  Xaa is a variable which may be A,R,N,D,C,Q,E,G,H,I,L,K,M,F,P,S,T,W,Y,V This is a synthetically prepared peptide
ESXV

SEQ ID NO: 3 - protein, 4 aa, Artificial Sequence
  Xaa at position 2 is phosphorylated serine or phosphorylated threonine This is a synthetically prepared peptide
EXXV

SEQ ID NO: 4 - protein, 12 aa, Artificial Sequence
  Xaa is phosphorylated serine This is a synthetically prepared peptide
HRRAARYLEXAV

SEQ ID NO: 5 - protein, 16 aa, Artificial Sequence
  This is a synthetically prepared peptide
HRRSARYLESSVDAEF

SEQ ID NO: 6 - protein, 16 aa, Artificial Sequence
  This is a synthetically prepared peptide
HRRSARYLAASVDAEF

SEQ ID NO: 7 - protein, 13 aa, Artificial Sequence
  This is a synthetically prepared peptide
HRRSARYLESSVR

SEQ ID NO: 8 - protein, 12 aa, Artificial Sequence
  This is a synthetically prepared peptide
HRRSARYLESSV

SEQ ID NO: 9 - protein, 12 aa, Artificial Sequence
  Xaa is phosphorylated serine This is a synthetically prepared peptide
HRRAARYLEXAV

SEQ ID NO: 10 - protein, 12 aa, Artificial Sequence
  This is a synthetically prepared peptide
HRRSARYLAASV

SEQ ID NO: 11 - protein, 4 aa, Artificial Sequence
  Xaa at position 2 is a variable which may be T or S This is a synthetically prepared peptide
DXXL

SEQ ID NO: 12 - protein, 4 aa, Artificial Sequence
  Synthetic peptide
STWM

SEQ ID NO: 13 - protein, 3 aa, Artificial Sequence
  XAA in position 1 is F or Y This is a synthetically prepared peptide
XXX

SEQ ID NO: 14 - protein, 3 aa, Artificial Sequence
  synthetic peptide
YYA

SEQ ID NO: 15 - protein, 4 aa, Artificial Sequence
  Xaa in position 2 is phophorylated threonine or phosphorylated serine This is a synthetically prepared peptide
DXXL

SEQ ID NO: 16 - protein, 4 aa, Artificial Sequence
  Xaa in position 1 is phosphorylated serine or S This is a synthetically prepared peptide
XXWM

SEQ ID NO: 17 - protein, 3 aa, Artificial Sequence
  Xaa in position 1 is phosphorylated tyrosine This is a synthetically prepared peptide
XXX

SEQ ID NO: 18 - protein, 3 aa, Artificial Sequence
  Xaa in position 1 is a variable which may be phosphorylated tyrosine or Y This is a synthetically prepared peptide
XXA

SEQ ID NO: 19 - protein, 5 aa, Artificial Sequence
  Xaa in position 2 is a variable which may be T or S This is a synthetically prepared peptide
EXXVX

SEQ ID NO: 20 - protein, 5 aa, Artificial Sequence
  Xaa in position 2 is a variable which may be T or S
DXXLX

SEQ ID NO: 21 - protein, 5 aa, Artificial Sequence
  Xaa is a variable which may be A,R,N,D,C,Q,E,G,H,I,L,K,M,F,P,S,T,W,Y,V
STWMX

SEQ ID NO: 22 - protein, 4 aa, Artificial Sequence
  Xaa at position 1 is a variable which may be F or Y
XXXX

SEQ ID NO: 23 - protein, 4 aa, Artificial Sequence
  Xaa is a variable which may be A,R,N,D,C,Q,E,G,H,I,L,K,M,F,P,S,T,W,Y,V
YYAX

SEQ ID NO: 24 - protein, 16 aa, Artificial Sequence
  synthetic peptide
HRRSARYLDTVLEEMS

SEQ ID NO: 25 - protein, 11 aa, Artificial Sequence
  synthetic peptide
HRRSARYLDTV

SEQ ID NO: 26 - DNA, 26 bp, Artificial Sequence
  primer
cgggatccctgccccggctctgctgc

SEQ ID NO: 27 - DNA, 31 bp, Artificial Sequence
  primer
ggaattccagctgctcgtccgtctcggggtc

(19) United States
(12) Patent Application Publication
(10) Pub. No.: US 2005/0164311 A1
(43) Pub. Date: Jul. 28, 2005

(54) Biological assay detection method

(76) Inventors: James Inglese, Lansdale, PA (US); Marc Ferrer, Media, PA (US); Aaron C. Hamilton, Hatfield, PA (US)

Correspondence Address: MERCK AND CO., INC, P O BOX 2000, RAHWAY, NJ, 07065-0907, US

(21) Appl. No.: 10/484,859

(22) PCT Filed: Aug. 2, 2002
(86) PCT No.: PCT/US02/24469

Related U.S. Application Data
(60) Provisional application No. 60/310,599, filed on Aug. 7, 2001.

(51) Int. Cl. G01N 33/53; C07K 7/08; C07K 7/06

(52) U.S. Cl. 435/7.5; 530/408

(57) ABSTRACT

The invention is a method for detecting a reaction product which signals the presence of a reaction product inducer such as an enzyme. The method enables the recognition of epitopes that form the basis of a detection strategy without the need for specific antibodies to the epitope. In the method, a directly or indirectly labeled modular domain and a biotinylated form of the cognate peptide ligand are used as the basis for a measurable interaction. The peptide ligand can be masked by modifications through, for example, phosphorylation of the Ser or Thr residue, or extension of the amino acid sequence beyond the C-terminal Val. Because the masked residues are critical to binding of the labeled modular domain, masking of at least one of the residues prevents binding. Upon treatment of the masked residue by the appropriate enzyme, (e.g., treatment of the phosphorylated residue with a phosphatase enzyme, or treatment of the extended residue with a protease enzyme, the peptide is converted to the original unmasked ligand that is capable of binding to the labeled modular domain and forming a measurable complex.

### BACKGROUND OF THE INVENTION

For decades, immunological methods have formed the basis for detection strategies in wide-ranging medical and biological applications. Variations of the enzyme-linked immunosorbent assay, ELISA, have been employed for clinical diagnostic tests and in drug discovery programs of the pharmaceutical industry. The advent of the monoclonal antibody and subsequent work leading to the development of monoclonal antibody library repertoires from recombinant and display technologies (Gao, C., et al., (1999) Proc. Natl. Acad. Sci. USA 96(11): 6025-6030) have further leveraged the power of these selective high affinity proteins in many areas of science.

While innovations in antibody technology promise to expand access to these reagents for assay development, an alternative approach would build on the inherent specificity and accessibility of the diversity of protein/peptide interactions found in nature, such as those comprising cellular signaling systems. Enzymes, receptors and adaptor proteins such as Grb2 and PSD95 found within many signal transduction networks often contain one or more modular domains (e.g., SH2, SH3, PTB and PDZ domains) responsible for mediating molecular interactions frequently through peptide-protein associations (Pawson, T. N., et al., Genes & Development 14(9): (2000) pp. 1027-1047). Some of these domain-ligand combinations have the potential to function in much the same way as antibody-antigen pairs in assay detection systems.

### SUMMARY OF THE INVENTION

The invention is a method for detecting a reaction product which signals the presence of a reaction product inducer such as an enzyme. The method enables the recognition of epitopes that form the basis of a detection strategy without the need for specific antibodies to the epitope.

In the method, a directly or indirectly labeled modular domain and a biotinylated form of the cognate peptide ligand are used as the basis for a measurable interaction. The peptide ligand can be masked by modifications through, for example, phosphorylation of the Ser or Thr residue, or extension of the amino acid sequence beyond the C-terminal Val. Because the masked residues are critical to binding of the labeled modular domain, masking of at least one of the residues prevents binding. Upon treatment of the masked residue by the appropriate enzyme, (e.g., treatment of the phosphorylated residue with a phosphatase enzyme, or treatment of the extended residue with a protease enzyme, the peptide is converted to the original unmasked ligand that is capable of binding to the labeled modular domain and forming a measurable complex.

### DETAILED DESCRIPTION OF THE INVENTION AND PREFERRED EMBODIMENTS

The invention is a method for detecting, under suitable conditions, the presence, in a sample, of a complex inducer that converts a modified ligand incapable of forming a complex with a directly or indirectly labeled modular domain into a ligand capable of forming a complex with the labeled modular domain comprising a) combining the modified ligand, the labeled modular domain, and the sample, to form a combination, and b) analyzing the combination to detect the complex, indicating the presence in the sample of the complex inducer, wherein the detection conditions convert the modified ligand only in the presence of the complex inducer.

In a class of the method, the ligand is selected from the group consisting of

| | |
|---|---|
| biotin-(XX)₀₋₁₀ Glu-Thr-XX₁-Val- | (SEQ. ID No. 1) |
| COOH, | |
| biotin-(XX)₀₋₁₀ Glu-Ser-XX₁-Val- | (SEQ. ID No. 2) |
| COOH, | |
| biotin-(XX)₀₋₁₀ Asp-Thr-XX₁-Leu- | (SEQ. ID No. 11) |
| COOH, | |
| biotin-(XX)₀₋₁₀ Asp-Ser-XX₁-Leu- | (SEQ. ID No. 11) |
| COOH, | |
| biotin-(XX)₀₋₁₀ Ser-Thr-Trp-Met- | (SEQ. ID No. 12) |
| COOH, | |
| biotin-(XX)₀₋₁₀ XX₂-XX₁-Phe-COOH, | (SEQ. ID No. 13) |
| biotin-(XX)₀₋₁₀ XX₂-XX₁-Val-COOH, | (SEQ. ID No. 13) |
| biotin-(XX)₀₋₁₀ XX₂-XX₁-Ala-COOH, | (SEQ. ID No. 13) |
| biotin-(XX)₀₋₁₀ Tyr-Tyr-Ala-COOH, | (SEQ. ID No. 14) |

wherein
each XX, same or different, is an amino acid selected from the group consisting of Ala, Arg, Asn, Asp, Cys, Gln, Glu, Gly, His, Ile, Ieu, Lys, Met, Phe, Pro, Ser, Thr, Trp, Tyr, and Val;
XX₁ is an amino acid selected from the group consisting of Ala, Arg, Asn, Asp, Cys, Gln, Glu, Gly, His, Ile, Leu, Lys, Met, Phe, Pro, Ser, Thr, Trp, Tyr, and Val; and XX₂ is Phe or Tyr.

In a subclass of this class, the modified ligand is selected from the group consisting of

| | |
|---|---|
| biotin-(XX)₀₋₁₀ Glu-pThr-XX₁-Val- | (SEQ. ID No. 3) |
| COOH, | |
| biotin-(XX)₀₋₁₀ Glu-pSer-XX₁-Val- | (SEQ. ID No. 3) |
| COOH, | |
| biotin-(XX)₀₋₁₀ Asp-pThr-XX₁-Leu- | (SEQ. ID No. 15) |
| COOH, | |
| biotin-(XX)₀₋₁₀ Asp-pSer-XX₁-Leu- | (SEQ. ID No. 15) |
| COOH, | |
| biotin-(XX)₀₋₁₀ pSer-Thr-Trp-Met- | (SEQ. ID No. 16) |
| COOH, | |
| biotin-(XX)₀₋₁₀ Ser-pThr-Trp-Met- | (SEQ. ID No. 16) |
| COOH, | |
| biotin-(XX)₀₋₁₀ pTyr-XX₁-Phe-COOH, | (SEQ. ID No. 17) |
| biotin-(XX)₀₋₁₀ pTyr-XX₁-Val-COOH, | (SEQ. ID No. 17) |
| biotin-(XX)₀₋₁₀ pTyr-XX₁-Ala-COOH, | (SEQ. ID No. 17) |
| biotin-(XX)₀₋₁₀ pTyr-Tyr-Ala-COOH, | (SEQ. ID No. 18) |
| biotin-(XX)₀₋₁₀ Tyr-pTyr-Ala-COOH, | (SEQ. ID No. 18) |
| biotin-(XX)₀₋₁₀ Glu-Thr-XX₁-Val- | (SEQ. ID No. 19) |
| (XX₃)₁₋₄-NH₂, | |
| biotin-(XX)₀₋₁₀ Glu-Ser-XX₁-Val- | (SEQ. ID No. 19) |
| (XX₃)₁₋₄-NH₂, | |
| biotin-(XX)₀₋₁₀ Asp-Thr-XX₁-Leu- | (SEQ. ID No. 20) |
| (XX₃)₁₋₄-NH₂, | |
| biotin-(XX)₀₋₁₀ Asp-Ser-XX₁-Leu- | (SEQ. ID No. 20) |
| (XX₃)₁₋₄-NH₂, | |
| biotin-(XX)₀₋₁₀ Ser-Thr-Trp-Met- | (SEQ. ID No. 21) |
| (XX₃)₁₋₄-NH₂, | |
| biotin-(XX)₀₋₁₀ XX₂-XX₁-Phe- | (SEQ. ID No. 22) |
| (XX₃)₁₋₄-NH₂, | |
| biotin-(XX)₀₋₁₀ XX₂-XX₁-Val- | (SEQ. ID No. 22) |
| (XX₃)₁₋₄-NH₂, | |
| biotin-(XX)₀₋₁₀ XX₂-XX₁-Ala- | (SEQ. ID No. 22) |
| (XX₃)₁₋₄-NH₂, | |
| biotin-(XX)₀₋₁₀ Tyr-Tyr-Ala- | (SEQ. ID No. 23) |
| (XX₃)₁₋₄-NH₂, | |

wherein
each XX, same or different, is an amino acid selected from the group consisting of Ala, Arg, Asn, Asp, Cys, Gln, Glu, Gly, His, Be, Leu, Lys, Met, Phe, Pro, Ser, Thr, Trp, Tyr, and Val;
XX₁ is an amino acid selected from the group consisting of Ala, Arg, Asn, Asp, Cys, GIn, Glu, Gly, His, Ile, Leu, Lys, Met, Phe, Pro, Ser, Thr, Trp, Tyr, and Val; and
each XX₃, same or different, is an amino acid selected from the group consisting of Ala, Arg, Asn, Asp, Cys, Gln, Glu, Gly, His, ile, Ieu, Lys, Met, Phe, Pro, Ser, Thr, Trp, Tyr, and Val.

In a group of this subclass, the labeled modular domain is labeled with labeled chelate, e.g., Eu³⁺ chelate, a labeled antibody, e.g., Eu³⁺ antibody, or labeled colloidal particles.

In a subgroup of this group, the labeled modular domain is selected from the group consisting of labeled PDZ domain, labeled SH2 domain, labeled SH3 domain, and labeled PIB domain.

In a division of this subgroup, the complex inducer is selected from the group of enzymes consisting of phosphatase, protease, kinase, hydrolase and deacetylase enzyme.

The invention is a time-resolved fluorescence resonance energy transfer detection method based on ligands of a modular domain developed for enzymatic assays as an alternative to immuno-based detection strategies. The peptide enzyme substrate is a masked domain ligand with the consensus sequence Ser-XX₁-Val-OH. The critical residues in the binding consensus sequence of the ligand have been modified, for example by phosphorylation of Ser and C-terminal extensions, providing binding incompetent modular domain peptides. Upon processing by the corresponding enzyme, the binding epitope is exposed, and the product sequence is recoged specifically by the modular domain which is labeled, e.g., with labeled chelate such as Eu³⁺ chelate, a labeled antibody such as Eu³⁺ antibody, or a labeled colloidal particle. A complex is then formed by addition of streptavidin labeled with an allophycocyanin which binds to the biotin at the N-terminus of the peptide, and detected by time resolved fluorescence resonance energy transfer.

### Abbreviations

ALPHA Amplified luminescent proximity homogeneous assay
BSA bovine serum albumin
CPB carboxypeptidase B
DELFIA dissociation enhanced lanthanide fluoroimmunoassay
DMSO dimethyl sulfoxide
GST-PDZ glutathione-S-transferase fused to PDZ domain
GST-PDZ1 glutathione-S-transferase fused to PDZ1 domain

HTRF homogeneous time resolved fluorescence
ITC isothiocyanate
PBS phosphate buffered saline
PBST phosphate buffered saline with tween-20
PBSTB phosphate buffered saline with tween-20 and BSA
PCI protein carboxypeptidase inhibitor
PP2A₁ protein phosphatase 2A₁
SH2 src homology 2
SH3 src homology 3
PTB phosphotyrosine binding
PDZ PSD95/Discs-large/ZO-1
PSD95 postsynaptic density 95
TR-FRET time resolved fluorescence resonance energy transfer
[XL665]SA XL665 streptavidin
XL665 modified allophycocyanin

A “complex inducer” is an enzyme, e.g. porcine carboxypeptidase B, protein phosphatase 2A₁, which induces formation of a complex which is the ligand bound to the labeled modular domain.

The “complex” is the ligand bound to the labeled modular domain.

The “ligand” is the entity that binds to the labeled modular domain to form the complex when acted on by the complex inducer, and which does not bind to the labeled modular domain when not acted on by the complex inducer.

The “modified ligand” is the ligand which does not bind to the labeled modular domain.

The “modular domain” is a sequence (e.g. SH2, SH3, PmB and PDZ) responsible for mediating molecular interactions through peptide-protein associations including peptide-protein binding or interaction (see Harrison Cell vol. 86 pp. 341-343 (1996) and Pawson et al. Genes & Development 14: 1027-1047 (2000)).

The “labeled modular domain” is a modular domain which enables detection of the complex formed by the ligand and the labeled modular domain.

“Suitable detection conditions” are conditions which, in the absence of the complex inducer, do not promote conversion of the modified ligand incapable of forming a complex with a labeled modular domain to the ligand capable of forming a complex with the labeled modular domain.

Biotin-peptides were purchased from Research Genetics (Huntsville, Ala.) or Princeton Biomolecules (Langhorne, Pa.). Peptides were dissolved in DMSO to ˜1 mM solutions (concentration was determined by UV absorbance at 280 nm, using a ε_{Tyr}=1280 mol⁻¹Lcm⁻¹). Porcine carboxypeptidase B and potato carboxypeptidase inhibitor were from Sigma (St. Louis, Mo.). Protein phosphatase 2A₁(PP2A₁), cantharidic acid and okadaic acid were from Biomol Research Laboratories (Plymouth Meeting, Pa.). Asp-N was from Roche (Germany). Streptavidin-XL665 (molar ratio: 1.9 XL665/Streptavidin) was from Packard (Meriden, Conn.).

Assays were carried out in 96-well black, low binding, Microfluor2 plates (Dynex, Va.). Assay plates were read using a Victor²V microplate analyzer (Perkin Elmer Wallac (Turku, Finland)), with 337 nm excitation. Fluorescence emission was measured at 665 nm for FRET signal and 615 nm for Eu³⁺ chelate, and results are expressed as the ratio of fluorescence intensities, FI₆₆₅ₙₘ/FI_{615 nm}.

Modular domains can be labeled using labeled chelate, antibody or colloidal particles. Labeled chelate could be, for example, Eu³⁺ chelate, labeled antibody could be, for example, Eu³⁺ antibody available from PerkinElmerWallac. Labeled colloidal particles could be, for example, such particles available from Packard BioScience and known as ALPHA detection technology, a commonly used read-out method (http://www.packardbioscience.com/products/298.asp).

Modular domain ligands can be masked by phosphorylation of Ser, Thr or Tyr, if present in the ligand (e.g., phosphorylating Ser to form pSer), or by using sequence extensions beyond the C-terminus.

Labeling of GST-PDZ with Eu³⁺ Chelate

The GST-PDZ domain fusion protein corresponding to the PDZ module 3 of PSD-95 (Songyang, Z. et al. Science 275: (1997) pp. 73-77) was obtained by standard cloning and expression methods. Briefly, the PSD-95 (including amino acids Leu343-Ala445) coding sequence was amplified from a rat brain cDNA library (Clontech, Inc.) using PCR, and subcloned into the bacterial expression vector, pGEX2KT (Pharmacia), GST-PDZ was expressed in E. coli BL21(DE3) using standard bacterial expression methods (Smith and Johnson Gene 67: (1988) pp. 31-40), and purified using reduced glutathione agarose beads (Molecular Probes) following protocols provided by the supplier. GST-PDZ was labeled with Eu³⁺-chelate ITC (Perkin Elmer Wallac), as described by the manufacturers. Briefly, GST-PDZ was concentrated to about 3 mg/ml in PBS, and 50 μl (160 μg, 4.3 nmol) of protein solution added to lyophilized Eu³⁺ chelate ITC (100 μg, 140 nmol), and reaction let to proceed for 16 hours at 4° C. The reaction solution was applied to a Nick column (Amersham Pharmacia), and the fraction with labeled protein (400 μl in PBS) applied to a PD-10 column (Amersham Pharmacia). Labeled protein was eluted in 400 μl fractions with PBS. Fractions with protein were pooled. Protein concentration was quantified by Bradford Protein assay (Pierce, Ill.), and protein bound Eu³⁺ chelate was quantified with DELIA enhancement solution for Eu³⁺, and Eu³⁺ standard (Perkin Elmer Wallac). Final protein concentration was 0.32 mg/ml (9 mM, 5-fold molar ratio Eu³⁺/protein.

Detection Conditions

Detection conditions giving suitable signal-to-background ratio for each enzymatic system were established by titrating the amount of [Eu³⁺ ]GST-PDZ and [XL665]SA simultaneously, at a constant concentration of biotin-peptide. XL665 is a modified allophycocyanin acceptor fluorophore which emits at 665 nm with a slow decay time. The detection conditions were optimed at a total biotin-peptide concentration of 40 nM, assuming a 10% turnover, that is 4 nM biotin-product peptide and 36 nM biotin-substrate peptide for each enzymatic system. 10 μl of 5× HIRF mixture were added to 40 μl of biotin-peptide solution. Concentrations are those in the final 50 μl assay volume. Optimal incubation time of the peptide with the detection reagents was found to be 1 hr.

To determine the dynamic range of the detection and the lower detection limit, the amount of biotin-product peptide was increased keeping the total amount of peptide constant at 40 nM. As described above, 10 μl of 5× HUB mixture were added to 40 μl of biotin-peptide solution. Concentrations are those in the final 50 μl assay volume. The signal was measured using the concentrations of detection reagents determined to be optimal as described in the previous section.

GST-PDZ1 Indirectly Labeled with Eu³⁺ Antibody

Subcloning and Expression of GST-PDZ Domains. The GST-PDZ domain fusion protein corresponding to the PDZ module 1 of rabbit NHERF was obtained by standard cloning and expression methods. Briefly, the PDZ1 NHERF (including amino acids leu11 to leu99) coding sequence was amplified from a plasmid of containing the DNA sequence of the PDZ1 of rat NHERF using PCR with the following primers: 5′-CGGGATCCCTGCCCCGGCTCTGCTGC (SEQ. ID No. 26) and 5′-GGAATTCCAGCTGCTCGTCCGTCTCGGGGTC (SEQ. ID No. 27). The DNA was subcloned into the bacterial expression vector pGEX-2TK Pharmacia) using the BaMHI and EcoRI restrictions sites. The GST-PDZ was expressed in E. Coli BL21(DE3) using standard bacterial expression methods (Smith and Johnson Gene 67: (1988) pp. 31-40), and purified using reduced glutathione agarose beads (Molecular Probes, Eugene, Oreg.) following protocols provided by the supplier.

Assay conditions. Binding of the PDZ1 domain of NHERF to several peptide ligands was also investigated using indirect labeling of the PDZ1 domain with Eu³⁺. In this approach, described in Hemmila, I. W. S. (1997) Drug Discovery Today 2(9); 373-381 and Kolb, et al. (1998) Drug Discovery Today 3; 333-342, we employed the GST-PDZ described earlier bound to an anti-GST labeled with Eu³⁺ chelate (Perkin Elmer Wallac). The Eu³⁺ labeled anti-GST allowed us to label the GST-PDZ1 without structurally modifying the PDZ1 domain itself (avoiding direct coupling to free Lys in the domain). In order to test binding of the peptides, 10 nM peptide and 10 nM GST-PDZ domain were incubated together for 1 hr (20 uL PBSTB). After incubation, a detection mixture comprised of the labeled components of the assay were added. Due to the 1:1 interaction of GST-fusion proteins and the anti-GST antibody, 10 nM anti-GST(Eu³⁺) was used and due to the tetrameric nature of streptavidin, 2.5 nM of [XL665]SA was used (20 uL PBSTB). The final mix was incubated at room temperature and read on the Victor 2. The indirect labeling of the NHERF GST-PDZ1 produced a labeled protein that did bind to the potential peptide ligands tested.

GST-PDZ1-Peptide Ligand Binding Measured with ALPHA

ALPHA technology (see Glickman et al. (2002) J. Biomolecular Screening 7(1): 3-10) was also investigated as a means to detect binding of potential peptide ligands to NHERF GST-PDZ1. This assay, based on the proximity of a donor bead (which releases singlet oxygen) and acceptor bead (that fluoresces in the presence of singlet oxygen), involved using the previously described GST-PDZ1. In order to probe the interaction of the biotinylated peptides and the GST-PDZ1, streptavidin donor beads were used (to capture the biotinylated peptide) and anti-GST labeled acceptor beads were used (to capture the GST-PDZ1). If an interaction between the peptide and the GST-PDZ1 occurred, the acceptor beads would fluoresce when read on the ALPHAquest reader. In order to test the binding of the peptides, 10 nM biotin-peptide and 10 nM GST-PDZ1 were incubated for 1 hr (PBSTB). In order to determine the optimal concentration of streptavidin donor beads, the amount of streptavidin donor beads (5 uL in PBSTB) was titrated at a constant concentration of anti-GST acceptor beads (20 ug/mL) (5 uL). 10 uL of the peptide/GST-PDZ1 mix was added to the detection mix containing the donor and acceptor beads. The plates were sealed and incubated in the dark at room temperature for 3 hours before being read on the ALPHAquest reader. The use of the ALPHA detection reagents allowed for an assay that reproduced the results (showed binding of the potential peptide ligands to GST-PDZ1) from the indirect labeling of the GST-PDZ1, only yielding higher signal/background.

### EXAMPLE 1

Carboxypeptidase B Assay

For the initial time courses, 20 μl of biotin-HRRSARYLESSVR-OH (SEQ. ID No. 7), (100 nM in 10 mM phosphate, 150 mM NaCl, 0.05% tween-20, 0.1% BSA (PBSTB)) was mixed with 20 μl of CPB (2× in PBSTB), and the reaction quenched with 10 μl of a 5×TR-FET mixture of PCI (1 μM, [Eu³⁺]GST-PDZ (6.25 nM), and [X1665]SA (62.5 nM) in PBSTB, for 60 min, at 25° C., and read.

For the inhibition experiments, 2 Ill of inhibitor was added to 20 μl CPB (200 pM in PBSTB), and the reaction was initiated by addition of 20 μl peptide substrate (100 nM in PBSTB). The reaction was quenched after 30 min with the addition of 10 μl of 5×TR-FRET quenching/detection mixture, as described above. Background was measured from a reaction mixture with substrate, detection mixture and no enzyme.

### EXAMPLE 2

Protein Phosphatase Assay

For the initial time courses, 20 μl of biotin-HRRAARYLEpSAV-OH (SEQ. ID No. 4) (100 nM in phosphatase buffer: 50 mM Tris, pH 7.4, 10% glycerol, 14 mM β-mercaptoethanol, 0.2 mg/ml BSA) was mixed with 20 μl of PP2A₁ (2× in phosphatase buffer), and the reaction solution was incubated at 32° C. At different time points, 40 μl aliquots were quenched with 10 W of a 5×TR-FRET mixture of Cantharidic Acid (5 μM, [Eu³⁺]GST-PDZ (1.6 nM), and [665]SA (15.6 nM) in PBSTB, for 60 min, at 25° C., and read.

For the inhibition experiments, 2 μl of inhibitor were added to 20 μl PP2A₁ (30 pM in phosphatase buffer), and the reaction was initiated by addition of 20 μl of peptide substrate (100 nM in phosphatase buffer). The reaction was quenched after 60 min with the addition of 10 μl of 5×TR-FRET quenching/detection mixture, as described above. Background was measured from a reaction mixture with substrate, detection mixture and no enzyme.

Table 1 shows results obtained in Examples 1 and 2, including the enzyme tested, enzyme concentration, enzyme hydrolysis rate, IC₅₀ values, and assay conditions, e.g. enzyme dilution or direct read of a “tracer”. The hydrolysis rate allows for identification of a time point at which a measurement can be made and assumed to be in the linear range for the purpose of estimating concentration of the target enzyme.

TABLE 1

| Enzyme | [Enzyme] | Initial Rate | Condition | Inhibitor | IC₅₀ |
|---|---|---|---|---|---|
| PP2A₁ | 15 pM | 0.001 pmol | Dilution | Okadaic Acid | 15 pM |
| | | product/min | | | |
| | | 0.001 pmol | Dilution | Cantharidic | 56 nM |
| | | product/min | | Acid | |
| CPB | 100 pM | 0.005 pmol | Dilution | PCI | 10 nM |
| | | product/min | | | |
| Asp-N | 20 nM | 0.0015 pmol | Dilution | None tested | N/A |
| | | product/min | | | |
| | 20 nM | 0.0007 pmol | Tracer | None tested | N/A |
| | | product/min | | | |

### EXAMPLE 3

Asp-N Protease Assay

For the initial time courses, 20 μl of biotin-HRRSARYLESSVDAEF-NH₂ (SEQ. ID No. 5) (20 μM in PBST) was mixed with 20 μl of Asp-N (2× in PBST), and the reaction solution was incubated at 32° C. At different time points, aliquots were diluted 1/200 in PBST, and 10 W of the 5× detection mixture ([Eu³⁺] GST-PDZ (6.25 nM), and [XL665]SA (15.6 nM) in PBST) was added to 40 μl of the diluted solution, incubated for 60 min, at 25° C., and measured.

For the trace experiment, 20 μl of a mixture of biotin-HRRSARYLESSVDAEF-NH₂ (100 nM) and HRRSARYLAASVDAEF-NH₂ (SEQ. ID No. 6)(20 μM) in PBST, were added to 20 μl of Asp-N (40 nM in PBST), and the reaction was incubated at 32° C. At a different time points, 40 W aliquots were quenched with 10 μl of a 5× quenching/detection mixture (50 mM EDTA, [Eu³⁺]GST-PDZ (6.3 nM), and [XL665]SA (15.6 nM) in PBST), for 60 min, at 25° C., and measured.

### EXAMPLE 4

HIV Protease Assay

Using biotin-HRRSARYLDTVLEEMS-OH (SEQ. ID No. 24), Eu³⁺-labeled GST antibody and a procedure similar to Example 1, an assay was performed to identify the presence of H[V protease.

Design of Masked Modular Domain Ligands

The use of fluorescence resonance energy transfer (FRET) for measuring enzymatic activities in a homogeneous format relies on the specific recognition of the enzymatic product by fluorescently labeled reporter molecules. The invention includes peptide substrates for specific enzymes that once processed, bind to a fluorescently labeled PDZ domain reporter protein. Two, proteases, porcine carboxypeptidase B and Asp-N, and a Ser/Thr phosphatase, PP2A₁, were tested.

The critical residues in the PDZ binding consensus sequence of the ligand (-Glu-Ser/Thr/X-Val-OH (SEQ. ID No. 1 and SEQ. ID No. 2) were modified, for example by C-terminal extensions (for porcine carboxypeptidase B and Asp-N assays) and phosphorylation of the critical Ser (for PP2A₁ assay), providing enzyme substrates that are binding-incompetent PDZ domain peptides. Table 2 shows the pairs of peptide sequences.

TABLE 2

| | |
|---|---|
| Substrate | Product |

| | |
|---|---|
| Enzyme—Porcine carboxypeptidase B | |
| Biotin-HRRSARYLESSVR-OH | Biotin-HRRSARYLESSV-OH |
| (SEQ. ID No. 7) | (SEQ. ID No. 8) |
| Enzyme-Asp-N | |
| Biotin-HRRSARYLESSVDAEF-NH₂ | Biotin-HRRSARYLESSV-OH |
| (SEQ. ID No. 5) | (SEQ. ID No. 8) |
| Enzyme-Phosphatase 2A₁ (PP2A₁) | |
| Biotin-HRRAARYLEpSAV-OH | Biotin-HRRAARYLESAV-OH |
| (SEQ. ID No. 9) | (SEQ. ID No. 9) |
| Enzyme-Asp-N (tracer experiment) | |
| Biotin-HRRSARYLESSVDAEF-NH₂/ | Biotin-HRRSARYLESSV-OH/ |
| HRRSARYLAASVDAEF-NH₂ | HRRSARYLAASV-OH |
| | (SEQ. ID No. 10) |
| Enzyme-HIV protease | |
| Biotin-HRRSARYLDTVLEEMS-NH₂ | Biotin-HRRSARYLDTV-OH |
| (SEQ. ID No. 24) | (SEQ. ID No. 25) |

The requirement of a specific C-terminal residue on PDZ ligands presents the opportunity for the design of novel protease assays. The addition of an arginine to the C-terminus of HRRSARYLESSV-OH results in a peptide substrate for carboxypeptidase (Folk, J. E. Methods Enzymology vol. 19 (1970) p. 504). By extending the sequence of amino acids beyond a single additional amino acid residue, substrates for different endoproteases can be made. Asp-N shows specificity for Asp in the P₁′ position. Other proteases, such as HIV protease, prefer hydrophobic residues at the P₁-P₁′, surrounding the scissile bond, Glu or Gln at the P₂′ site and small residues at the P₂ site (Erickson et al. Proteases of Infectious Agents B. M. Dunn, San Diego Academic Press (1999) pp. 1-60), all potentially compatible as a pro-PDZ ligand.

A second residue in the peptide ligand critical for binding to the PSD95 PDZ-3 is Ser of the X-Glu-Ser-X-Val-COOH (SEQ. ID No. 2) binding motif in HRRSARYLESSV-OH. Masking of this residue by phosphorylation, for example, disrupts PDZ binding until the peptide is dephosphorylated by a Ser/Thr phosphatase. In this phosphatase detection scheme, Ser-₁, a ligand residue not critical to binding, was changed to alanine to simplify the synthesis of the phosphoserine peptide (HRRAARYLEpSAV-OH).

Time Resolved Fluorescence Resonance Energy Transfer Detection Reagents

The TR-FRET system used here is based on FRET occurring between the long-lived Eu³⁺-chelate energy donor and the modified allophycocyanin energy acceptor, [XL655]SA (Kolb et al. DDT vol. 3 no. 7 Jul. 1998 pp. 333-342). Donor-acceptor FRET signaling pairs include, but are not limited to, Eu³⁺-tribipyridine cryptate/allophycocyanin (XL 665), Tb³⁺-cryptate/rhodamine, and EDANS (5-(2′-aminoethyl)aminonaphthalene sulfonic acid/DABCYL (4-(4′-dimethyl aminobenzineazo)benzoic acid. Time resolved fluorometry is more generally described by Hemmila et al., DDT vol. 2, no. 9 Sep. 1997 pp. 373-381.

The feasibility of the detection strategy was first tested by measuring a FRET signal at varying concentrations of [Eu³⁺]GST-PDZ and [665]SA in the presence of 4 nM of a PDZ peptide ligand (reaction product) plus 36 nM of a ‘masked’ form of the peptide ligand (reaction substrate). The peptide concentrations are representative of a mixture containing 10% product peptide and 90% substrate peptide, designed to mimic 10% substrate turnover of 50 nM total biotinylated peptide in a 40 μl assay volume. Addition of 10 μl of a 5×TR-FRET detection mixture was then added to allow formation of the FRET signaling complex. Three substrate/product systems were studied corresponding to three different enzymatic reactions described above. The results indicate that for each of the peptide pairs there is an optimal set of Eu³⁺] GST-PDZ and [XL665]SA concentrations for which the signal-to-background is maximum (at least 10-fold). For example, biotin-HRRAARYLEpSAV-OH/biotin-HRRAARYLESAV-OH corresponding to the phosphatase reaction, the optimal concentrations of [Eu³⁺] GST-PDZ and [XL665]SA were determined to be 0.3 nM and 3.1 nM, respectively. Similar experiments were conducted for each peptide substrate/product combination designed giving similar results (data not shown). These optimal concentrations of [Eu³⁺] GST-PDZ and [XL165]SA were subsequently used when studying each one of the enzymatic reactions.

1. A method for detecting, under suitable conditions, the presence, in a sample, of a complex inducer that converts a modified ligand incapable of forming a complex with a directly or indirectly labeled PDZ domain into a ligand capable of forming a complex with the labeled PDZ domain comprising a) combining the modified ligand, the labeled PDZ domain, and the sample, to form a combination, and b) analyzing the combination to detect the complex, indicating the presence in the sample of the complex inducer, wherein the detection conditions convert the modified ligand only in the presence of the complex inducer.

2. A method of claim 1 wherein the ligand is selected from the group consisting of
biotin-(XX)0-10 Glu-Thr-XX₁-Val-COOH,
biotin-(XX)0-10 Glu-Ser-XX₁-Val-COOH,
biotin-(XX)0-10 Asp-Thr-XX₁-Leu-COOH,
biotin-(XX)0-10 Asp-Ser-XX₁-Leu-COOH,
biotin-(XX)0-10 Ser-Thr-Trp-Met-COOH,
biotin-(XX)0-10 XX₂-XX₁-Phe-COOH,
biotin-(XX)0-10 XX₂-XX₁-Val-COOH,
biotin-(XX)0-10 XX₂-XX₁-Ala-COOH, and
biotin-(XX)0-10 Tyr-Tyr-Ala-COOH,
wherein
each XX, same or different, is an amino acid selected from the group consisting of Ala, Arg, Asn, Asp, Cys, Gln, Glu, Gly, His, Ile, Leu, Lys, Met, Phe, Pro, Ser, Thr, Trp, Tyr, and Val;
XX₁ is an amino acid selected from the group consisting of Ala, Arg, Asn, Asp, Cys, Gln, Glu, Gly, His, Ile, Leu, Lys, Met, Phe, Pro, Ser, Thr, Trp, Tyr, and Val; and XX₂ is Phe or Tyr.

3. A method of claim 1 wherein the modified ligand is selected from the group consisting of
biotin-(XX)0-10 Glu-pThr-XX₁-Val-COOH,
biotin-(XX)0-10 Glu-pSer-XX₁-Val-COOH,
biotin-(XX)0-10 Asp-pThr-XX₁-Leu-COOH,
biotin-(XX)0-10 Asp-pSer-XX₁-Leu-COOH,
biotin-(XX)0-10 pSer-Thr-Trp-Met-COOH,
biotin-(XX)0-10 Ser-pThr-Trp-Met-COOH,
biotin-(XX)0-10 pTyr-XX₁-Phe-COOH,
biotin-(XX)0-10 pTyr-XX₁-Val-COOH,
biotin-(XX)0-10 pTyr-XX₁-Ala-COOH,
biotin-(XX)0-10 pTyr-Tyr-Ala-COOH,
biotin-(XX)0-10 Tyr-pTyr-Ala-COOH,
biotin-(XX)0-10 Glu-Thr-XX₁-Val-(XX₃)1-4-NH₂,
biotin-(XX)0-10 Glu-Ser-XX₁-Val-(XX₃)1-4-NH₂,
biotin-(XX)0-10 Asp-Thr-XX₁-Leu-(XX₃)1-4-NH₂,
biotin-(XX)0-10 Asp-Ser-XX₁-Leu-(XX₃)1-4-NH₂,
biotin-(XX)0-10 Ser-Thr-Trp-Met-(XX₃)1-4-NH₂,
biotin-(XX)0-10 XX2—XX1-Phe-(XX₃)1-4-NH₂,
biotin-(XX)0-10 XX2—XX1-Val-(XX₃)1-4-NH₂,
biotin-(XX)0-10 XX2—XX₁-Ala-(XX₃)1-4-NH₂, and
biotin-(XX)0-10 Tyr-Tyr-Ala-(XX₃)1-4-NH₂,
wherein
each XX, same or different, is an amino acid selected from the group consisting of Ala, Arg, Asn, Asp, Cys, Gln, Glu, Gly, His, Ile, Leu, Lys, Met, Phe, Pro, Ser, Thr, Trp, Tyr, and Val;
XX₁ is an amino acid selected from the group consisting of Ala, Arg, Asn, Asp, Cys,
Gln, Glu, Gly, His, Ile, Leu, Lys, Met, Phe, Pro, Ser, Thr, Trp, Tyr, and Val; and each XX₃, same or different, is an amino acid selected from the group consisting of
Ala, Arg, Asn, Asp, Cys, Gln, Glu, Gly, His, Ile, Leu, Lys, Met, Phe, Pro, Ser, Thr, Trp, Tyr, and Val.

4. A method of claim 1 wherein the labeled PDZ domain is labeled with labeled chelate.

5. A method of claim 4 wherein the labeled chelate is Eu³⁺ chelate.

6. A method of claim 1 wherein the labeled PDZ domain is labeled antibody.

7. A method of claim 6 wherein the labeled antibody is Eu³⁺ antibody

8. A method of claim 1 wherein the labeled PDZ domain is labeled with labeled collodial particles.

9. (canceled)

10. A method of claim 1 wherein the complex inducer is selected from the group of enzymes consisting of phosphatase, protease, kinase, hydrolase and deacetylase.
